(19) United States
(12) Patent Application Publication
(10) Pub. No.: US 2012/0264928 A1
(43) Pub. Date: Oct. 18, 2012

(54) THERAPEUTIC PHARMACEUTICAL AGENT FOR DISEASES ASSOCIATED WITH DECREASE IN FUNCTION OF GNE PROTEIN, FOOD COMPOSITION, AND FOOD ADDITIVE

(75) Inventors: Satoru Noguchi, Tokyo (JP); May Christine Malicdan, Tokyo (JP); Ichizo Nishino, Tokyo (JP)

(73) Assignee: JAPAN HEALTH SCIENCES FOUNDATION, Tokyo (JP)

(21) Appl. No.: 13/320,320

(22) PCT Filed: May 14, 2010
(86) PCT No.: PCT/JP2010/058116

§ 371 Date: Jun. 29, 2012

(30) Foreign Application Priority Data
May 15, 2009 (JP) 2009-119272

(51) Int. Cl. C07H 7/02; C07H 5/06

(52) U.S. Cl. 536/53; 536/18.7

(57) ABSTRACT

Disclosed are a therapeutic pharmaceutical agent for diseases associated with the decrease in the function of GNE protein, a food composition, and a food additive. The therapeutic pharmaceutical agent is characterized by comprising a compound capable of increasing the quantity of N-acetylneuraminic acid in cells. Examples of the compound to be contained in the therapeutic pharmaceutical agent include N-acetylneuraminic acid, an intermediate produced downstream from N-acetylmannosamine in an N-acetylneuraminic acid biosynthesis pathway, an N-acetylneuraminic acid derivative, an N-acetylmannosamine derivative, an N-acetylneuraminic acid-containing compound, an N-acetylneuraminic acid derivative-containing compound, an N-acetylmannosamine-containing compound, an N-acetylmannosamine derivative-containing compound, an inhibitor of a degrading enzyme for N-acetylneuraminic acid, an inhibitor of a degrading enzyme for N-acetylmannosamine, an inhibitor of a degrading enzyme for the intermediate, and others.

### CROSS-REFERENCE TO RELATED APPLICATIONS

The present application claims the priority of Japanese Patent Application No. 2009-119272 filed on May 15, 2009, which is incorporated herein by reference.

### TECHNICAL FIELD

The present invention relates to therapeutic pharmaceutical agents, food compositions, or food additives for use in diseases caused by decrease of a GNE protein function.

### BACKGROUND ART

Among myopathies (muscular diseases), distal myopathy with rimmed vacuoles (DMRV) and hereditary inclusion body myopathy (HIBM) are known to occur by the loss-of-function mutation of a GNE gene and are autosomal recessive diseases with an age of onset in the range of 15 to 40 years.

The GNE gene codes for a UDP-GlcNAc2-epimerase/ManNAc kinase, which is a rate-limiting enzyme for an N-acetylneuraminic acid biosynthetic pathway (see, for example, Non-patent Literatures 1 and 2). This enzyme plays a role in two enzymatic reactions from UDP-GlcNAc to ManNAc and from ManNAc to ManNAc6-phosphate. Thus, it has been reported that the amount of N-acetylneuraminic acid decreases in skeletal muscle cells affected by myopathy and their primary cultured cells (see, for example, Noguchi, S. et al., J. Biol. Chem., 279(12), 11402-11407, 2004; and Nonaka, I. et al., Curr. Neurol. Neurosci. Rep., 5(1), 61-65, 2005).

The pathological characteristics of a muscle tissue affected by myopathy caused by a mutation of the GNE gene include the formation of rimmed vacuoles, irregular sized muscle fibers, the formation of an intranuclear inclusion body, and β-amyloid protein deposition. Clinicopathologically, the tibialis anterior muscle is particularly likely to be affected, and the cervical flexor muscle, the paraspinal muscle, and the knee flexor muscle on the posterior surface of the thigh are also likely to be affected. With the progress of the disease, a muscle group on the posterior surface of the leg and the upper limb muscle are also affected, but the quadriceps femoris muscle is not affected before a relatively late stage.

A process through which myopathy caused by a mutation of the GNE gene results in muscular atrophy is not clear. Thus, it is desirable to elucidate the process and develop an effective therapeutic method or an effective therapeutic agent.

However, many findings have been reported that deny the possibility of the therapeutic administration of N-acetylneuraminic acid to patients. For example, it has been reported that, because of its acidity, an N-acetylneuraminic acid molecule is difficult to incorporate into cells of animals having GNE gene mutation and normal animals (see, for example, Datta, Biochemistry 13, 3987-3991, 1978; Harms and Reutter, Cancer Res., 34, 3165-3172, 1974; Hirschberg et al., Biochemistry 15, 3591-3599, 1976; Diaz and Varki, Anal. Biochem., 150, 32-46, 1985; and Ferwerda et al., Biochem. Soc. Transactions 17, 744-745, 1989). Furthermore, it has been reported that N-acetylneuraminic acid has a very short half-life in the blood of animals (see, for example, Nohle, U. et al., Eur. J. Biochem., 126, 543-548, 1982), and the administration of free N-acetylneuraminic acid has no particular effect of increasing N-acetylneuraminic acid in ganglioside (see, for example, Carlson, S. E. and House, S. G., J. Neutr., 116, 881-886, 2009). Thus, it is believed that the administration of N-acetylneuraminic acid as a pharmaceutical agent has limited clinical efficacy. Thus,
N-acetylneuraminic acid has not been studied as an active substance of a pharmaceutical agent (see, for example, WO 2008/150477 A2).

### SUMMARY OF INVENTION

### Technical Problem

It is an object of the present invention to provide a therapeutic pharmaceutical agent, a food composition, or a food additive for use in myopathy caused by decrease of a GNE protein function.

### Solution to Problem

A pharmaceutical agent according to the present invention is a therapeutic pharmaceutical agent for a disease caused by decrease of a GNE protein function and contains one or more compounds selected from the group consisting of N-acetylneuraminic acid, an intermediate produced downstream of N-acetylmannosamine in an N-acetylneuraminic acid biosynthetic pathway, an N-acetylneuraminic acid derivative, an N-acetylmannosamine derivative, a compound containing N-acetylneuraminic acid, a compound containing an N-acetylneuraminic acid derivative, a compound containing N-acetylmannosamine, a compound containing an N-acetylmannosamine derivative, an N-acetylneuraminic acid degrading enzyme inhibitor, an N-acetylmannosamine degrading enzyme inhibitor, and an inhibitor of a degrading enzyme of the intermediate.

The N-acetylneuraminic acid derivative has the following formula 1:

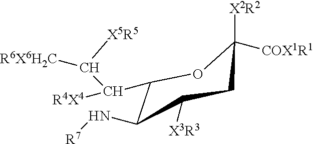

wherein X^{P} (P is an integer from 1 to 6) denotes O or S, R^{P} (P is an integer from 2 to 6) denotes hydrogen, an alkanoyl, or an alkyl in the case that X^{P} adjacent to the R^{P} is O or denotes an alkanoyl or an alkyl in the case that X^{P} adjacent to the R^{P} is S, R¹ denotes hydrogen, an alkyl, or an alkanoylalkyl in the case that X′ is O or denotes an alkyl or an alkanoylalkyl in the case that X′ is S, and R⁷ denotes hydrogen, an alkanoyl, or a hydroxyalkanoyl.

The N-acetylmannosamine derivative has the following formula 2:

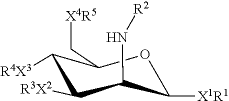

wherein X^{P} (P is an integer from 1 to 4) denotes O or S, R^{P} (P is an integer selected from 1, 3, 4, and 5) denotes hydrogen, an alkyl, an alkanoylalkyl, or an alkanoyl in the case that X^{P} adjacent to the R^{P} is O or denotes an alkyl, an alkanoylalkyl, or an alkanoyl in the case that X^{P} adjacent to the R^{P} is S, and R² denotes hydrogen or an alkanoyl.

Preferably, the alkanoyl, alkyl, alkanoylalkyl, or hydroxyalkanoyl in the formulae 1 and 2 is a lower one.

More preferably, the loss of the GNE protein function is caused by a mutation of the GNE gene. Still more preferably, the disease is renal dysfunction or myopathy.

Preferably, the intermediate is N-acetylmannosamine-6-phosphate or N-acetylneuraminic acid-9-phosphate.

Preferably, the N-acetylneuraminic acid derivative is Ac5NeuAc or Ac5NeuAc-Me, and the N-acetylmannosamine derivative is Ac4ManNAc.

Preferably, the compound containing N-acetylneuraminic acid is sialyllactose.

The inhibitor of the degrading enzyme of the intermediate is preferably GlcNAcol or a GlcNAcol derivative, and the GlcNAcol derivative is more preferably Ac5GlcNAcol.

A food composition according to the present invention contains one or more compounds selected from the group consisting of an N-acetylneuraminic acid derivative, an N-acetylmannosamine derivative, an N-acetylneuraminic acid degrading enzyme inhibitor, an N-acetylmannosamine degrading enzyme inhibitor, and an inhibitor of the degrading enzyme of an intermediate produced downstream of N-acetylmannosamine in an N-acetylneuraminic acid biosynthetic pathway.

The N-acetylneuraminic acid derivative has the following formula 1:

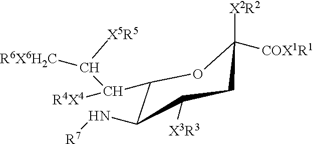

wherein X^{P} (P is an integer from 1 to 6) denotes O or S, R^{P} (P is an integer from 2 to 6) denotes hydrogen, an alkanoyl, or an alkyl in the case that X^{P} adjacent to the R^{P} is O or denotes an alkanoyl or an alkyl in the case that X^{P} adjacent to the R^{P} is S, R¹ denotes hydrogen, an alkyl, or an alkanoylalkyl in the case that X′ is O or denotes an alkyl or an alkanoylalkyl in the case that X′ is S, and R⁷ denotes hydrogen, an alkanoyl, or a hydroxyalkanoyl.

The N-acetylmannosamine derivative has the following formula 2:

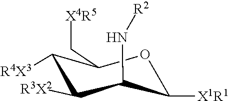

wherein X^{P} (P is an integer from 1 to 4) denotes O or S, R^{P} (P is an integer selected from 1, 3, 4, and 5) denotes hydrogen, an alkyl, an alkanoylalkyl, or an alkanoyl in the case that X^{P} adjacent to the R^{P} is O or denotes an alkyl, an alkanoylalkyl, or an alkanoyl in the case that X^{P} adjacent to the R^{P} is S, and R² denotes hydrogen or an alkanoyl.

Preferably, the alkanoyl, alkyl, alkanoylalkyl, or hydroxyalkanoyl in the formulae 1 and 2 is a lower one.

Preferably, the N-acetylneuraminic acid derivative is Ac5NeuAc or Ac5NeuAc-Me, and the N-acetylmannosamine derivative is Ac4ManNAc.

The inhibitor of the degrading enzyme of the intermediate is preferably GlcNAcol or a GlcNAcol derivative, and the GlcNAcol derivative is more preferably Ac5GlcNAcol.

A food according to the present invention contains any of the food compositions described above.

A food additive according to the present invention contains one or more selected from N-acetylneuraminic acid, an intermediate produced downstream of N-acetylmannosamine in an N-acetylneuraminic acid biosynthetic pathway, and a compound containing N-acetylneuraminic acid, wherein the N-acetylneuraminic acid, the intermediate produced downstream of N-acetylmannosamine in the N-acetylneuraminic acid biosynthetic pathway, and the compound containing N-acetylneuraminic acid are compounds purified from natural products or chemically synthesized compounds.

Preferably, the total amount of the N-acetylneuraminic acid, the intermediate produced downstream of N-acetylmannosamine in the N-acetylneuraminic acid biosynthetic pathway, and the compound containing N-acetylneuraminic acid is 50% or more of the food additive.

A method for producing a food containing a food additive according to the present invention includes the addition of any of the food additives described above.

A food containing a food additive according to the present invention is produced by any of the methods for producing a food containing a food additive described above.

### BRIEF DESCRIPTION OF DRAWINGS

FIG. 1 shows photomicrographs of myotubes derived from DMRV model mice, which were cultured in the presence of various reagents and were labeled with desmin, WGA, or SBA, according to an embodiment of the present invention.

FIG. 2 is a graph of the amount of NeuAc in myotubes derived from a human DMRV patient and cultured in the presence of various reagents of different concentrations according to an embodiment of the present invention.

FIG. 3 is a graph of the amount of NeuAc in myotubes derived from DMRV model mice and cultured in the presence of various reagents according to an embodiment of the present invention.

FIG. 4 is a graph of the survival rates of DMRV model mice treated with various reagents according to an embodiment of the present invention.

FIG. 5 is a graph of the survival rates of DMRV model mice treated with various reagents according to an embodiment of the present invention.

FIG. 6 is a graph of the amount of NeuAc in muscle tissues of DMRV model mice treated with various reagents according to an embodiment of the present invention.

FIG. 7 is a graph of the amount of NeuAc in muscle tissues of DMRV model mice treated with 40 or 400 mg/kg Ac4ManNAc according to an embodiment of the present invention.

FIG. 8 is a graph of the blood creatine kinase activity of DMRV model mice treated with various reagents according to an embodiment of the present invention.

FIG. 9 is a graph of the running distances of DMRV model mice treated with various reagents according to an embodiment of the present invention.

FIG. 10 is a graph of the running distances of DMRV model mice treated with various reagents according to an embodiment of the present invention.

FIG. 11 is a graph of the hanging times of DMRV model mice treated with various reagents according to an embodiment of the present invention.

FIG. 12 is a graph of the times of the electrical stimulation given to DMRV model mice treated with various reagents in an endurance test according to an embodiment of the present invention.

FIG. 13 is a graph of the times of the electrical stimulation given to DMRV model mice treated with various reagents in an endurance test according to an embodiment of the present invention.

FIG. 14 is a graph of the cross-sectional area of a gastrocnemius muscle of DMRV model mice treated with various reagents according to an embodiment of the present invention.

FIG. 15 is a graph of the specific contractile force of a gastrocnemius muscle of DMRV model mice treated with various reagents according to an embodiment of the present invention.

FIG. 16 is a graph of the specific contractile force of a gastrocnemius muscle of DMRV model mice treated with various reagents according to an embodiment of the present invention.

FIG. 17 is a graph of Pₜ (isometric contractile force)/the cross-sectional area of a muscle of DMRV model mice treated with various reagents according to an embodiment of the present invention.

FIG. 18 is a graph of Pₜ (isometric contractile force)/the cross-sectional area of a muscle of DMRV model mice treated with various reagents according to an embodiment of the present invention.

FIG. 19 shows photomicrographs of muscle tissues of DMRV model mice treated with various reagents and subjected to hematoxylin-eosin staining (H-E), acid phosphatase activity staining, anti-amyloid antibody (LC3) labeling, or congo red staining according to an embodiment of the present invention.

FIG. 20 shows photomicrographs of muscle tissues of DMRV model mice treated with various reagents and labeled with an anti-β-amyloid antibody (Aβ1-40 or Aβ1-42) or an anti-phosphorylated tau antibody according to an embodiment of the present invention.

FIG. 21 is a graph of the number of rimmed vacuoles in muscle tissues of DMRV model mice treated with various reagents according to an embodiment of the present invention.

FIG. 22 is a graph of the number of rimmed vacuoles containing amyloid in muscle tissues of DMRV model mice treated with various reagents according to an embodiment of the present invention.

FIG. 23 shows photomicrographs of muscle tissues of DMRV model mice treated with 40 or 400 mg/kg Ac4ManNAc and subjected to hematoxylin-eosin staining (H-E), Gomori trichrome staining, or acid phosphatase activity staining according to an embodiment of the present invention.

FIG. 24 shows photomicrographs of muscle tissues of DMRV model mice treated with 40 or 400 mg/kg Ac4ManNAc and labeled with an anti-Lamp2 antibody, an anti-β-amyloid antibody (Aβ1-42), or an anti-p62 antibody according to an embodiment of the present invention.

### DESCRIPTION OF EMBODIMENTS

Embodiments of the present invention based on these findings will be described in detail below with reference to examples. However, the present invention is not limited to these examples.

Unless otherwise specified in the embodiments and examples, methods described in standard protocols or their modifications or alternations will be used. The standard protocols include J. Sambrook, E. F. Fritsch & T. Maniatis (Ed.), Molecular cloning, a laboratory manual (3rd edition), Cold Spring Harbor Press, Cold Spring Harbor, N.Y. (2001); and F. M. Ausubel, R. Brent, R. E. Kingston, D. D. Moore, J. G. Seidman, J. A. Smith, K. Struhl (Ed.), Current Protocols in Molecular Biology, John Wiley & Sons Ltd. Unless otherwise specified, a commercially available reagent kit or measuring apparatus is used in accordance with the accompanying protocol.

The objects, features, advantages, and ideas of the present invention will be apparent to a person skilled in the art with reference to the present specification. A person skilled in the art can easily embody the present invention with reference to the present specification. In the embodiments and specific examples, preferred embodiments and examples of the present invention are illustrated by way of example and not by way of limitation. It will be apparent to a person skilled in the art that various modifications may be made to the preferred embodiments and examples without departing from the spirit and scope of the present invention.

### <Compound>

Compounds for use in the production of a pharmaceutical agent, a food composition, or a food additive according to the present invention will be described in detail below.

(1) N-acetylneuraminic acid

N-acetylneuraminic acid may be derived from any source, for example, natural N-acetylneuraminic acid isolated and purified by a well-known method from animal tissues, cultured cells, mammalian milk, or eggs containing N-acetylneuraminic acid, or chemically synthesized N-acetylneuraminic acid.

### (2) Intermediate Produced Downstream of N-Acetylmannosamine in N-Acetylneuraminic Acid Biosynthetic Pathway

Preferably, the intermediate produced downstream of N-acetylmannosamine in the N-acetylneuraminic acid biosynthetic pathway is N-acetylmannosamine-6-phosphate or N-acetylneuraminic acid-9-phosphate. The intermediate may be derived from any source, for example, a natural intermediate isolated and purified by a method well known to a person skilled in the art from animal tissues or cultured cells, or a chemically synthesized intermediate.

### (3) N-Acetylneuraminic Acid Derivative and N-Acetylmannosamine Derivative

The N-acetylneuraminic acid derivative has the following formula 1:

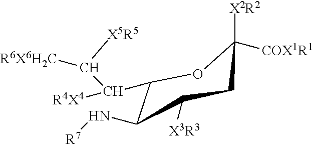

wherein X^{P} (P is an integer from 1 to 6) denotes O or S, R^{P} (P is an integer from 2 to 6) denotes hydrogen, an alkanoyl, or an alkyl in the case that X^{P} adjacent to the R^{P} is O or denotes an alkanoyl or an alkyl in the case that X^{P} adjacent to the R^{P} is S, R¹ denotes hydrogen, an alkyl, or an alkanoylalkyl in the case that X¹ is O or denotes an alkyl or an alkanoylalkyl in the case that X¹ is S, and R⁷ denotes hydrogen, an alkanoyl, or a hydroxyalkanoyl. More specifically, X^{P} adjacent to the RP is X¹, X², X³, X⁴, X⁵, or X⁶ for R¹, R², R³, R⁴, R⁵, or R⁶, respectively. The X^{P}s (P is an integer from 1 to 6) and the R^{P}s (P is an integer from 1 to 7) are independently selected.

The N-acetylmannosamine derivative has the following formula 2:

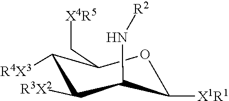

wherein X^{P} (P is an integer from 1 to 4) denotes O or S, R^{P} (P is an integer selected from 1, 3, 4, and 5) denotes hydrogen, an alkyl, an alkanoylalkyl, or an alkanoyl in the case that X^{P} adjacent to the R^{P} is O or denotes an alkyl, an alkanoylalkyl, or an alkanoyl in the case that X^{P} adjacent to the R^{P} is S, and R² denotes hydrogen or an alkanoyl. More specifically, X^{P} adjacent to the R^{P} is X¹ for R¹, X² for R³, X³ for R⁴, or X⁴ for R⁵. The X^{P}s (P is an integer from 1 to 4) and the R^{P}s (P is an integer from 1 to 5) are independently selected.

Preferably, the alkanoyl, alkyl, alkanoylalkyl, or hydroxyalkanoyl in the formulae 1 and 2 is a lower one.

Unless otherwise specified, the alkyl, alkoxy, alkenyl, alkynyl, or the like has both a straight chain and a side chain. A non-branched group, such as “propyl”, has a straight chain alone.

Although each of the R groups is specifically described below, the R groups are not limited to these examples. The lower alkyl is preferably a (C₁-C₆)alkyl, for example. More specifically, the lower alkyl or (C₁-C₆)alkyl may be methyl, ethyl, propyl, isopropyl, butyl, isobutyl, sec-butyl, pentyl, 3-pentyl, or hexyl. A (C₃-C₆)cycloalkyl may be cyclopropyl, cyclobutyl, cyclopentyl, or cyclohexyl. A (C₃-C₆)cycloalkyl(C₁-C₆)alkyl may be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl. A (C₂-C₆)alkenyl may be vinyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl. A (C₂-C₆)alkynyl may be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl. The lower alkanoyl is preferably a linear or branched (C₂-C₆)alkanoyl, for example, and more specifically may be acetyl, propanoyl, butanoyl, pentanoyl, or hexanoyl. A halo(C₁-C₆)alkyl may be iodomethyl, bromomethyl, chloromethyl, fluoromethyl, trifluoromethyl, 2-chloroethyl, 2-fluoroethyl, 2,2,2-trifluoroethyl, or pentafluoroethyl. A hydroxy(C₁-C₆)alkyl may be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl. A (C₁-C₆)alkoxycarbonyl may be methoxycarbonyl, ethoxycarbonyl, propoxycarbonyl, isopropoxycarbonyl, butoxycarbonyl, pentoxycarbonyl, or hexylcarbonyl. A (C₂-C₆)hydroxyalkanoyl may be glycolyl, lactyl, hydroxybutanoyl, hydroxypentanoyl, or hydroxyhexanoyl.

The N-acetylneuraminic acid derivative and the N-acetylmannosamine derivative may be derived from any source, such as a natural source, or may be a derivative synthesized by a method well known to a person skilled in the art. The synthetic N-acetylneuraminic acid derivative may be produced using any raw materials provided that a desired N-acetylneuraminic acid derivative can be synthesized. Examples of the raw materials include the N-acetylneuraminic acids described above and other N-acetylneuraminic acid derivatives synthesized by a well-known method. The synthetic N-acetylmannosamine derivative may be produced using any raw materials provided that a desired N-acetylmannosamine derivative can be synthesized. Examples of the raw materials include N-acetylmannosamine and other N-acetylmannosamine derivatives synthesized by a well-known method.

### (4) Compound Containing N-Acetylneuraminic Acid, Compound Containing N-Acetylneuraminic Acid Derivative, Compound Containing N-Acetylmannosamine, and Compound Containing N-Acetylmannosamine Derivative

The compound containing N-acetylneuraminic acid, the compound containing an N-acetylneuraminic acid derivative, the compound containing N-acetylmannosamine, and the compound containing an N-acetylmannosamine derivative may be any compound that contains N-acetylneuraminic acid, an N-acetylneuraminic acid derivative, N-acetylmannosamine, or an N-acetylmannosamine derivative as part of its structure, and may be sialyllactose, which is a natural saccharide containing N-acetylneuraminic acid, casein glycomacropeptide or mucin, which is a peptide containing N-acetylneuraminic acid, or ganglioside, which is a lipid containing N-acetylneuraminic acid. These compounds may be natural compounds or compounds artificially synthesized by a method well known to a person skilled in the art.

### (5) Degrading Enzyme Inhibitor

The N-acetylneuraminic acid degrading enzyme inhibitor, the N-acetylmannosamine degrading enzyme inhibitor, or the degrading enzyme inhibitor of an intermediate produced downstream of N-acetylmannosamine in the N-acetylneuraminic acid biosynthetic pathway may be any substance that can inhibit the degrading enzyme function of N-acetylneuraminic acid, N-acetylmannosamine, or the intermediate in cells. The N-acetylneuraminic acid degrading enzyme may be an N-acetylneuraminate pyruvate lyase. The N-acetylmannosamine degrading enzyme may be GlcNAc2-epimerase. The intermediate produced downstream of N-acetylmannosamine may be N-acetylmannosamine-6-phosphate or N-acetylneuraminic acid-9-phosphate. The degrading enzyme inhibitor may be any inhibitor that has an inhibitory action on the enzyme function and may be a silencing substance, such as siRNA that is specific for the DNA sequence coding for the enzyme. A desired siRNA may be synthesized by a method well known to a person skilled in the art. The inhibitor may be a compound that binds to an enzyme and inhibits the enzyme function. The N-acetylmannosamine degrading enzyme may be N-acetylglucosaminitol (GlcNAcol) or a GlcNAcol derivative. A suitable example of the GlcNAcol derivative may be a derivative having high cell permeability, such as acetylated N-acetylglucosaminitol (Ac5GlcNAcol). GlcNAcol and the GlcNAcol derivative may be synthesized by a method well known to a person skilled in the art and may be derived from any source. For example, an inhibitor of a bacteria-derived acetylneuraminate lyase (which is referred to as N-acetylneuraminate pyruvate lyase in mammals), which is a N-acetylneuraminic acid degrading enzyme, may be N-acetyl-4-oxo-neuraminic acid.

### <Method for Producing Pharmaceutical Agent>

A pharmaceutical agent according to the present invention contains one or more compounds selected from the group consisting of N-acetylneuraminic acid, an intermediate produced downstream of N-acetylmannosamine in an N-acetylneuraminic acid biosynthetic pathway, an N-acetylneuraminic acid derivative, an N-acetylmannosamine derivative, a compound containing N-acetylneuraminic acid, a compound containing an N-acetylneuraminic acid derivative, a compound containing N-acetylmannosamine, a compound containing an N-acetylmannosamine derivative, an N-acetylneuraminic acid degrading enzyme inhibitor, an N-acetylmannosamine degrading enzyme inhibitor, and an inhibitor of a degrading enzyme of the intermediate, described in the “Compounds”.

The formulation of a pharmaceutical agent according to the present invention involves the use of pharmaceutical additives well known to a person skilled in the art, such as a pharmaceutically acceptable carrier, diluent, and excipient. Any dosage form may be chosen that allows the pharmaceutical agent to be delivered to an affected part of a patient. For example, an oral preparation may be a tablet, a capsule, granules, a powder, syrup, an enteric coated preparation, a sustained-release capsule, cashew, a chewable tablet, a drop, a pill, an internal liquid medicine, a lozenge, a sustained-release tablet, or sustained-release granules. The dosage form may be an injection. The dosage form may be an external medicine, such as a poultice or ointment. A pharmaceutical agent according to the present invention may also be compounded with a different pharmaceutical composition, as well as the pharmaceutical additives described above.

### <How to Use Therapeutic Pharmaceutical Agent>

A therapeutic pharmaceutical agent according to the present invention can increase the amount of N-acetylneuraminic acid in cells in animals. Thus, a therapeutic pharmaceutical agent according to the present invention may also be used to treat or prevent any disease that is caused by a decrease in the amount of N-acetylneuraminic acid in cells, for example, a disease caused by decrease of a GNE protein function. The phrase “the decrease of a GNE protein function”, as used herein, refers to both total loss and partial loss of the function that GNE protein should have with respect to a target protein. The reason for the decrease of a GNE protein function is not particularly limited and may be unsuccessful expression of the GNE protein because of a difficulty in the expression process of the GNE protein, the degeneration of the protein structure after translation, resulting in malfunction of the GNE protein, or inhibition or modification that causes malfunction of the GNE protein. The reason may be a genetic factor, such as GNE gene mutation, or an external factor, such as an inhibitor. Examples of diseases caused by a mutation of the GNE gene include, but are not limited to, renal dysfunctions, such as glomerulonephritis, interstitial nephritis, nephronophthisis, and nephrotic syndrome, myopathy, and cardiomyopathy. A pharmaceutical agent according to the present invention may be administered to any animal, preferably humans or vertebrate animals other than humans.

A therapeutic pharmaceutical agent according to the present invention may be administered in a required amount within a safe dosage range by an appropriate method. The dose of pharmaceutical agent according to the present invention can finally be determined by a doctor or veterinarian in consideration of the dosage form, the mode of administration, and the age, body weight, and conditions of a subject, such as a patient.

### <Food Composition>

A food composition according to the present invention contains one or more compounds selected from the group consisting of an N-acetylneuraminic acid derivative, an N-acetylmannosamine derivative, an N-acetylneuraminic acid degrading enzyme inhibitor, an N-acetylmannosamine degrading enzyme inhibitor, and an inhibitor of the degrading enzyme of an intermediate produced downstream of N-acetylmannosamine in an N-acetylneuraminic acid biosynthetic pathway, described in the “Compounds”.

A food composition according to the present invention may be compounded with any desired component. For example, the component may be a vitamin, such as vitamin E or vitamin C, an emulsifier, a tonicity agent, a buffering agent, a solubilizing agent, a preservative, a stabilizer, or an antioxidant, or even a different food composition. A food composition according to the present invention may be used in any application, for example, as a food material for use in the production of a food, a dietary supplement, or a supplement, or as a food additive. A food containing the food composition may be produced by any method that can be appropriately selected by a person skilled in the art. The amount of food composition in the food is not particularly limited. A food containing a food composition according to the present invention may also contain a food additive described below.

### <Food Additive>

A food additive is used by means of addition, mixing, infiltration, or another method, in the production of a food or for the purpose of processing or preservation. A food additive according to the present invention contains one or more selected from N-acetylneuraminic acid, an intermediate produced downstream of N-acetylmannosamine in an N-acetylneuraminic acid biosynthetic pathway, and a compound containing N-acetylneuraminic acid, described in the “Compounds”. These compounds contained in a food additive according to the present invention are compounds purified from natural products or chemically synthesized compounds. The total amount of these compounds is preferably 50% or more, more preferably 70% or more, still more preferably 90% or more, of the food additive.

In addition to these compounds, a food additive according to the present invention may be compounded with any desired component. For example, the component may be a vitamin, such as vitamin E or vitamin C, an emulsifier, a tonicity agent, a buffering agent, a solubilizing agent, a preservative, a stabilizer, or an antioxidant, or even a different food additive. However, a component unsuitable for a food additive cannot be compounded with the food additive, for example, a component toxic for an animal that is to ingest a processed food containing the component at a certain concentration.

A food additive according to the present invention may be used in any application and may be added to a food produced by a method described below to produce a food containing the food additive.

### <Method For Producing Food Containing Food Additive>

A food containing a food additive according to the present invention is produced by the addition of the food additive according to the present invention in its production process. The addition of the food additive may be performed at any step of the food production process and may be appropriately determined by a person skilled in the art in a manner that depends on the type of the food. The amount of food additive to be added to the food can be determined by a person skilled in the art in consideration of the amount of food additive required for an animal that is to ingest the food and the intake of the food. Preferably, the food additive is added such that the concentration of N-acetylneuraminic acid, an intermediate produced downstream of N-acetylmannosamine in the N-acetylneuraminic acid biosynthetic pathway, or a compound containing N-acetylneuraminic acid is 10% or more of the food. A food containing a food additive according to the present invention may contain the food composition described above.

### <Food Containing Food Composition and Food Containing Food Additive>

A food containing a food composition or a food containing a food additive according to one embodiment of the present invention may be a general food, such as confectionery, a seasoning, a favorite food, or a drink. Specific examples include solid and semisolid favorite foods, such as cookies, biscuits, candies, gum, and jellies, favorite drinks, such as fruit juices, tea, coffee, and soft drinks, staple foods, such as breads and noodles, side dishes, such as soups, curries, stews, and various sauces, and various flavors and seasonings. A food according to the present invention may be a dietary supplement, a functional food, a food for specified health uses, or an enteral nutrient. The food may have the same dosage form as a pharmaceutical agent according to the present invention described above.

An animal that ingests a food composition according to the present invention or a food containing a food additive according to the present invention is not particularly limited and is preferably a human or a vertebrate animal other than humans, for example, a patient suffering from a disease to be treated with a pharmaceutical agent according to the present invention.

A human or a vertebrate animal other than humans can take a required amount of a food containing a food composition according to the present invention or a food containing a food additive according to the present invention as a folk medicine, a functional food, a health food, or a dietary supplement within the range of adequate intake levels. The intake of a food according to the present invention may be determined in consideration of the type of the food and the age and body weight of a subject that is to ingest the food. In the case that the subject suffers from a disease, the intake of a food according to the present invention is preferably determined in consideration of the type and the symptom of the disease. In these food applications, the food preferably has a label that indicates its effect. The label may indicate that the food is used to increase the amount of N-acetylneuraminic acid in cells or to alleviate a symptom of a disease caused by decrease of a GNE protein function. The label is not limited to these examples and may be any label that indicates the effect of the food.

### EXAMPLES

### Experimental Methods

### <DMRV Human Patient>

DMRV human patients are adult patients that have been diagnosed with DMRV by means of GNE gene mutation screening. (Number of patients: 42, the age of the onset of the disease: 20 to 30, sex: male and female) In accordance with a protocol approved by National Center of Neurology and Psychiatry, after obtaining informed consent, skeletal muscles (biceps brachii muscles and anterior tibial muscles) were collected from these patient volunteers under local anesthesia by means of biopsy.

### <DMRV Model Mice>

DMRV model mice were GNE^{(-/-)}hGNED176V-Tg described in Japanese Unexamined Patent Application Publication No. 2007-312641. Healthy litters having no GNE gene mutation were used as controls. The mice in the present example freely take water and feed and ingest 14 mg/kg body weight/day of N-acetylneuraminic acid compound on average.

### <Reagent>

N-acetylneuraminic acid (NeuAc) and N-acetylmannosamine (ManNAc) were purchased from Nacalai Tesque, Inc. Penta-O-acetyl-N-acetylneuraminic acid (Ac5NeuAc) and penta-O-acetyl-N-acetylneuraminic acid methyl ester (Ac5NeuAc-Me) were purchased from Nagara Science Co., Ltd. Sialyllactose (NeuAcα2-3Galβ1-4Glc) was purchased from Sigma-Aldrich Corp. Tetra-O-acetyl-N-acetylmannosamine (Ac4ManNAc) was purchased from NZP Ltd. Penta-O-acetyl N-acetylglucosaminitol (Ac5GlcNAcol) was synthesized from N-acetylglucosaminitol (purchased from Marker Gene Technologies) by a method according to Luchansky et al. (J. Biol. Chem., 278, 8035-8042, 2003). More specifically, 0.5 g of N-acetylglucosaminitol was dissolved in 5 ml of pyridine, 2.5 ml of acetic anhydride was added to the solution, and the solution was stirred overnight to cause a reaction. After the solvent was evaporated, the residue was dissolved in chloroform. The chloroform phase was then washed with 1.0 M hydrochloric acid, solid sodium hydrogen carbonate, and saturated saline. The chloroform phase was evaporated to dryness and was dissolved in ethanol. A product after HPLC was dissolved in ethanol and was stored at −20° C.

### <Preparation of Primary Cultured Cell of Myotube>

The skeletal muscle tissues collected from the DMRV human patients or the model mice were lightly washed with PBS or Hank's balanced saline and were treated with 0.25% trypsin for 5 minutes. The tissues were finely cut into several millimeters with ophthalmic scissors, were digested with 0.4% collagenase II/0.25% trypsin at 37° C. for 30 minutes, and were left still to collect supernatant. The precipitate was again treated with the enzyme liquid. After being left still, the precipitate was well suspended in a DMEM-Ham's F-12 culture medium. The tissue suspension was passed through a nylon mesh to remove the tissues. The washings and the supernatant were centrifuged to collect cells. The cells thus collected were plated on a DMEM-Ham's F-12 culture medium at a cell density of 10⁶ in a 100-mm plastic dish and were cultured at 37° C. in 5% CO₂ for 4 to 7 days.

### <Measurement of NeuAc and NeuGc Contents by HPLC>

In the case that the sample was cultured cells, the cells were washed with PBS three times, and 400 p. 1 of 50 mM sulfuric acid was added to the cells. The cells were incubated at 80° C. for 60 minutes and were hydrolyzed to release NeuAc and NeuGc. In the case that the sample was a piece of tissue, the tissue was frozen, was pulverized with an air hammer, and was homogenized in a KCl-tris solution. The precipitate was again washed with the KCl-tris solution and was hydrolyzed in 50 mM sulfuric acid at 80° C. for one hour to release NeuAc and NeuGc. 400 μl of 7 mmol 1,2-diamino-4,5-methylenedioxybenzene, dihydrochloride (MDB, Dojindo Laboratories) solution (15.8 mg MDB, 48.8 mg Na₂S₂O₄, and 735 μl 2-mercaptoethanol were dissolved in distilled water so as to prepare a 10-ml solution) was added to the sample containing free NeuAc and NeuGc and was allowed to react at 60° C. for 2.5 hours. The fluorescent derivative thus prepared was analyzed with HPLC (JASCO Corp.) using 0.05 to 5 nmol/μl Neu5Ac and Neu5Gc standards. The amount of protein from the tissue was measured with a Bio-Rad Protein Assay kit (Bio-Rad Laboratories).

### Example 1

The present example shows that NeuAc, a NeuAc derivative, and an intermediate produced downstream of ManNAc in the NeuAc biosynthetic pathway increase the amount of sialylated saccharide compound in a primary cultured cell of a myotube.

### <Administration of Reagent>

A reagent was added to the culture medium of primary cultured cells of myotubes derived from the DMRV model mice described above such that the final concentration was 5 mM ManNAc, 5 mM NeuAc, 5 mM Ac5NeuAc, 0.5 mM Ac5NeuAc-Me, or 0.2 mM Ac4ManNAc. The cells were cultured for additional three days.

### <Histological NeuAc Detection Method>

The cells cultured in the presence of the reagent were fixed with 4% paraformaldehyde at room temperature for 15 minutes and were treated with 0.05% saponin on ice for 30 minutes. The cells were detected with an anti-desmin antibody (catalog No. 69-181, ICN Pharmaceuticals), which is a myotube marker, and were counterstained with DAPI (Wako Pure Chemical Industries, Ltd.). The cells were incubated at room temperature for 30 minutes using an Alexa Fluor 568 labeled antibody (Invitrogen) as a secondary antibody. The cells were labeled with biotin-labeled SBA lectin (Seikagaku Corp.) or biotin-labeled WGA lectin (Seikagaku Corp.). The cells were incubated with FITC-labeled avidin (Vector Laboratories) at room temperature for 30 minutes to fluorescently label the biotin-labeled lectins. SBA recognizes a GalNAc structure in a sugar chain terminal structure. WGA recognizes a sialic acid cluster structure. This sialic acid contains NeuAc. The labeled primary cultured cells were observed with a confocal laser scanning fluorescence microscope (Olympus Corp.).

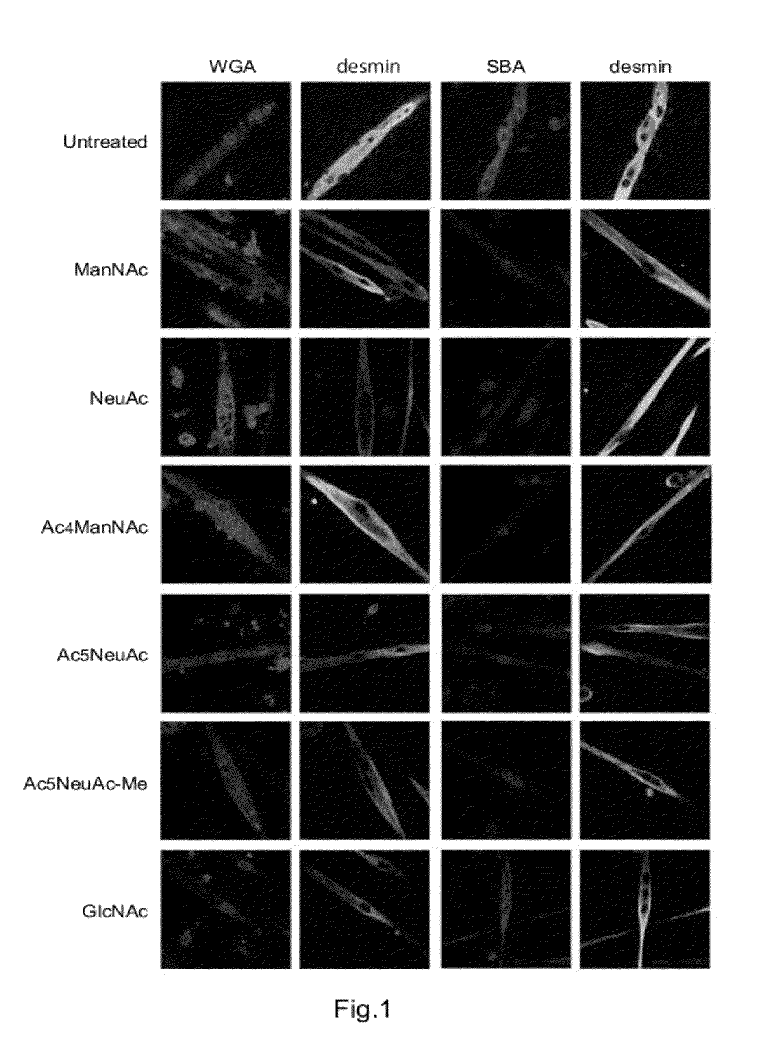

As shown in FIG. 1, in the primary cultured cells of an untreated group, desmin-positive myotubes were negative for WGA and positive for SBA. In these myotubes, sialic acid modification decreased and GalNAc modification increased in the sugar chain terminal structure. In contrast, in the myotubes in the culture media containing the reagents, WGA labeling increased and SBA labeling decreased as compared with the untreated group. In other words, sialic acid modification of the myotubes increased.

These results show that the reagents added to the culture media in the present example are effective in increasing the sialic acid modification of the sugar chain terminal structure in the myotubes derived from the DMRV model mice. Thus, these reagents are effective in increasing the sialic acid modification in cells.

### Example 2

The present example shows that NeuAc, a NeuAc derivative, a ManNAc derivative, and an intermediate produced downstream of ManNAc in the NeuAc biosynthetic pathway have a dose-dependent effect of increasing NeuAc.

### <Administration of Reagent>

ManNAc, NeuAc, or Ac5NeuAc was added to the culture media of primary cultured cells of myotubes derived from the DMRV human patients described above such that the final concentration was 0.005, 0.05, 0.5, or 5 mM. Ac4ManNAc, which has cytotoxicity when the Ac4ManNAc concentration is high, was added to the culture media such that the final concentration was 0.0002, 0.002, 0.02 or 0.2 mM. GalNAc was added to cells of a negative control group such that the final concentration was 0.005, 0.05, 0.5, or 5 mM. The cells were cultured for additional three days. The amount of NeuAc in the cultured cells was measured by the HPLC method described above (N=3).

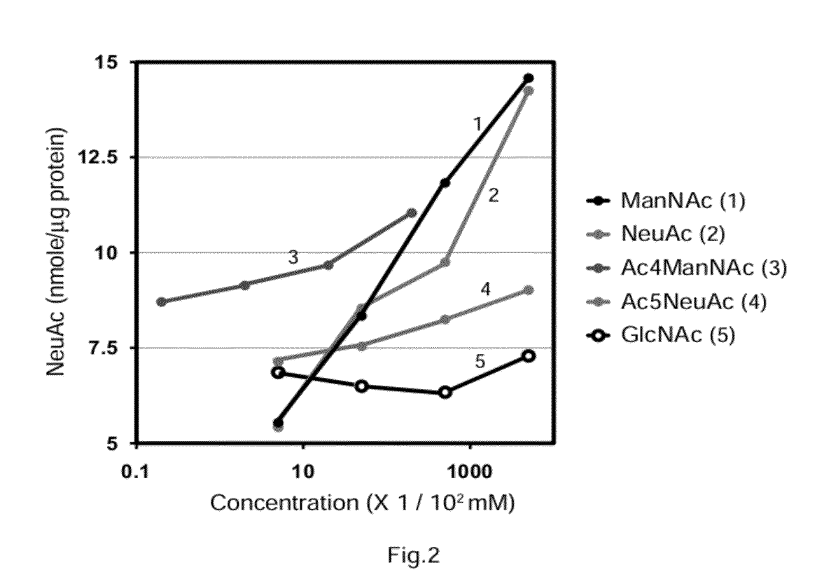

As shown in FIG. 2, ManNAc, NeuAc, and Ac5NeuAc were effective in increasing the amount of NeuAc in the cells in a manner that depended on their doses. Ac4ManNAc was also effective in increasing the amount of NeuAc at low concentrations in a manner that depended on its dose.

These results show that ManNAc, NeuAc, Ac5NeuAc, and Ac4ManNAc are effective in increasing the amount of NeuAc in the DMRV myotubes in a manner that depends on their doses.

### Example 3

The present example shows that a GalNAc2-epimerase inhibitor enhances the effect of ManNAc of increasing the amount of NeuAc.

### <Administration of Reagent>

ManNAc or Ac5GlcNAcol was added to the culture media of primary cultured cells of myotubes derived from the DMRV model mice such that the final concentration of ManNAc was 10 mM and the final concentration of Ac5GlcNAcol was 100 or 500 μm. The cells were then cultured for three days. 10 mM glucose (Glc) alone was added to a culture medium of cultured cells of a control group. The amount of NeuAc in the cultured cells was measured by the HPLC method described above (N=3).

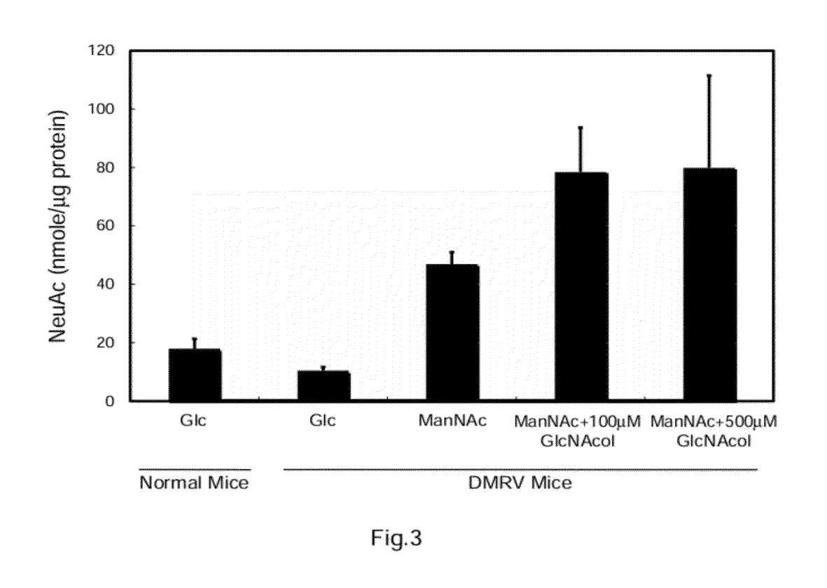

As shown in FIG. 3, in the myotubes derived from the DMRV model mice, the addition of ManNAc increased the amount of NeuAc, and the addition of 100 or 500 μm Ac5GlcNAcol together with ManNAc further increased the amount of NeuAc, as compared with the control group.

These results show that the intermediate of the NeuAc biosynthesis and the inhibitor of the degrading enzyme of the intermediate are effective in increasing the amount of NeuAc in the myotubes. These results also show that combined use of the intermediate and its degrading enzyme inhibitor enhances the effect of increasing NeuAc.

### Example 4

The present example shows that ManNAc, NeuAc, an intermediate of the NeuAc biosynthetic pathway, a NeuAc derivative, a ManNAc derivative, and a NeuAc-containing compound improve the conditions and survival rates of the DRVM model mice.

### <Administration of Pharmaceutical Agent>

20 mg/kg body weight/day of ManNAc (N=6), NeuAc (N=5), or sialyllactose (N=7) dissolved in drinking water was administered to the DMRV model mice between 11- to 15-week old and 56- to 58-week old for 43 to 45 weeks. 40 mg (N=5) or 400 mg/kg body weight/day (N=4) of Ac4ManNAc dissolved in drinking water was administered to the DMRV model mice of the same ages for 43 to 47 weeks. Drinking water free of the pharmaceutical agents was supplied to mice in a placebo group.

During the administration of the pharmaceutical agent, the measurement of the body weight, the determination of the survival rate, blood collection (0, 25, and 49 days after the start of administration, and thereafter at intervals of 28 days), and a hanging test (0 and 49 days after the start of administration, and thereafter at intervals of 56 days) were performed at regular intervals. Survivors after the administration (ManNAc-treated group: N=5, NeuAc-treated group: N=5, sialyllactose group: N=6) were subjected to a treadmill test. A muscle tissue was then excised and was subjected to a muscle contraction test, the measurement of the amount of NeuAc, and pathological observation.

### <Measurement of Blood Creatine Kinase Activity>

During the administration of the pharmaceutical agent, blood was collected from a tail of a mouse at regular intervals. The blood was centrifuged to prepare blood serum. Blood creatine activity was measured with a Determiner CPK-L kit (Kyowa Medex Co., Ltd.). The blood serum was electrophoresed using a Titan Gel Isoenzyme kit (Helen Laboratories) to identify creatine kinase. It is known that creatine kinase moves into blood when strenuous exercise or myopathy damages muscle fibers.

### <Hanging Test>

A wire net having a 6-mm lattice pattern made of wires having a diameter of approximately 0.5 mm was placed on a tube having a height of 50 cm. A mouse was made to hang on the wire net, and the time elapsed before the mouse fell down was measured. The time was measured three times for each mouse.

### <Treadmill Test>

In order to familiarize a mouse with the apparatus, training was started one week prior to testing. In this period, the mouse was made to run on a slope of 7 degrees at a velocity in the range of 5 to 15 m/min for 30 minutes every day for 7 days. As a motor ability test, the velocity was increased by 10 m/min per minute from the initial velocity of 20 m/min. The accumulated running distance up to the point where the mouse could not run anymore was measured. As an endurance test, a mouse was made to run on a slope of 7 degrees at a velocity of 20 m/min for 60 minutes. The mouse was then made to run for another 3 minutes, during which the times of the electrical stimulation given to the mouse by a stimulation grid disposed at the end of the running lane were measured.

### <Muscle Contraction Test>

After pentobarbital sodium (40 mg/kg body weight) was intraperitoneally administered to a mouse for anesthesia, a continuous muscle tissue of the tibialis anterior muscle and the gastrocnemius muscle was isolated. A terminal tendon of the isolated muscle tissue and the tibia were tied with a thread. The ends of the thread were connected at right angles to a tube for hanging a thread (a muscle length controller) and an isotonic transducer (TB-651T (for the gastrocnemius muscle) or TB-653TD-112S (for the tibialis anterior muscle), Nihon Kohden Corp.). The muscle tissue was placed in a lactated Ringer's solution (95% O₂, 5% CO₂). An electrostimulator (SEN-3301, Nihon Kohden Corp.) and an amplifier (PP-106H, Nihon Kohden Corp.) were used to expand the muscle tissue while providing 400 μs twitch stimulation. A length (L₀) at which the maximum contractile force was produced and the contractile force (isometric contractile force: Pₜ) at that length were measured. While the muscle tissue was held at the length (L₀) at which the contractile force was produced, the electrical stimulation was decreased to 3 ms. The maximum contractile force (P₀) was determined while 10- to 1000-Hz repetitive stimulation was provided 300 to 600 times at intervals of 2 minutes or more. After the measurement, the average cross-sectional area (CSA: muscle weight/L₀) of the muscle tissue was calculated.

### <Pathological Observation of Muscle Tissue>

The gastrocnemius muscle used in the muscle contraction test was frozen in isopentane cooled by liquid nitrogen, and a frozen section having a thickness of 6 μm was prepared with a cryostat. The section was placed on a slide glass. Hematoxylin-eosin (H-E) staining, acid phosphatase activity staining (see Malicdan et al., Method. Enzymol., 453, 379-396, 2009), or Gomori trichrome staining (see Malicdan et al., Method. Enzymol., 453, 379-396, 2009) were performed on adjacent sections. These sections were observed under an optical microscope. A frozen section having a thickness of 10 μm prepared in the same manner as described above was fixed with 4% paraformaldehyde, was stained with congo red, and was observed under a fluorescence microscope.

A frozen section having a thickness of 6 μm prepared in the same manner as described above was fixed with acetone and was blocked with a blocking solution (PBS containing 5% goat normal sera or 2% casein). The section was incubated with an anti-autophagy marker protein LC3 rabbit polyclonal antibody (NB100-2220, Novus Biologicals, 100 times dilution), an anti-β-amyloid rabbit polyclonal antibody (Aβ1-40, AB5074P, Chemicon, 100 times dilution), an anti-β-amyloid rabbit polyclonal antibody (Aβ1-42, AB5078P, Chemicon, 100 times dilution), an anti-phosphorylated tau mouse monoclonal antibody (90206, Innogenetics, 100 times dilution), an anti-amyloid mouse monoclonal antibody (6E10, Covance, 400 times dilution), an anti-p62 protein rabbit polyclonal antibody (PW9860, Biomol, 500 times dilution), or an anti-lysosome-associated membrane protein 2 (Lamp 2) rabbit polyclonal antibody (ABL-93, obtained from Developmental Studies Hybridoma Bank at the University of Iowa, 100 times dilution) at room temperature for one hour. If necessary, Alexa Fluor 488 or 568 labeled anti-rabbit/mouse IgG(H+L) (Molecular Probes) was used as a secondary antibody. Immunostained preparations of these sections were observed under a fluorescence microscope. The congo red staining and the LC3 labeling were performed on adjacent sections.

It is known that rimmed vacuoles found in muscle tissues of a patient suffering from myopathy are positive for acid phosphatase. DMRV skeletal muscle fibers have accumulated amyloid protein, and congo red recognizes amyloid and shows fluorescence. Tau protein is phosphorylated with amyloid β-protein. In myopathy caused by a mutation of the GNE gene, it is known that lysosomal vesicles that contain localized Lamp 2 protein are accumulated. It is also known that p62 protein recognizes polyubiquitin protein and directly binds to LC3 to induce autophagy in a portion that contains accumulated polyubiquitinated protein. In a muscle tissue affected by myopathy, p62 protein is colocalized with amyloid.

### Number of Formed Rimmed Vacuoles

In six H-E stained pathological sections having a thickness of 10 μm prepared at intervals of 100 μm, the number of rimmed vacuoles observed over the cross section of the muscular tissue was counted. In order to count the number of amyloid-positive protein deposits, the number of cells having deposits labeled with an anti-amyloid antibody (6E10) observed over the cross section of the muscular tissue was counted in six sections having a thickness of 10 μm prepared at intervals of 100 μm.

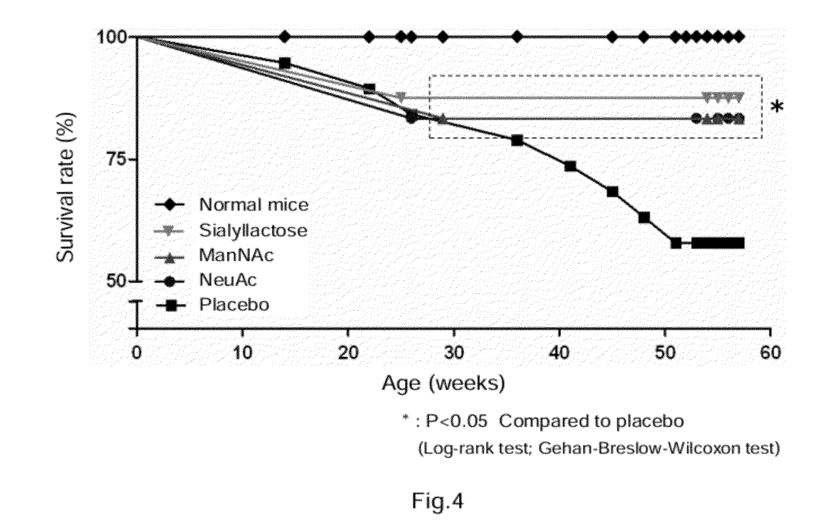

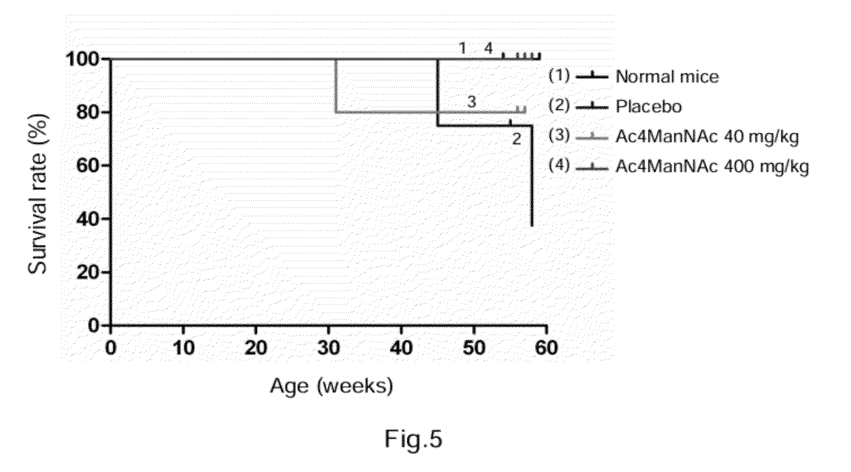

As shown in FIG. 4, the survival rates of the DMRV model mice were significantly higher in the ManNAc, NeuAc, and sialyllactose-treated groups than in the placebo group. As shown in FIG. 5, the survival rates of the DMRV model mice were also higher in the Ac4ManNAc-treated groups than in the placebo group.

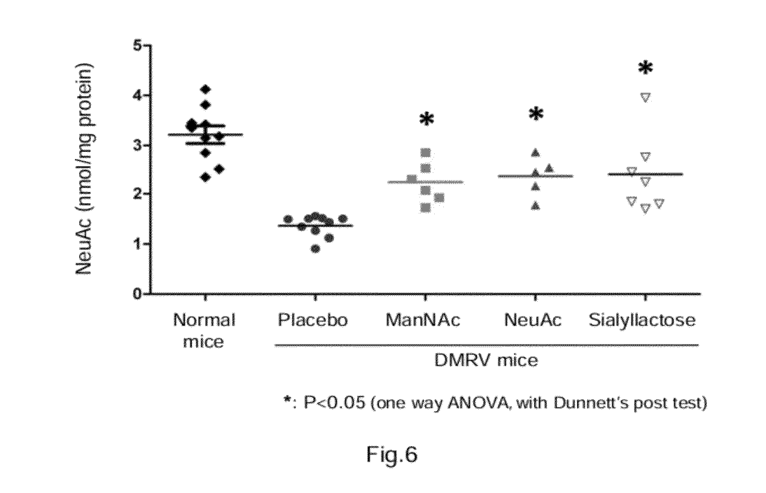

The amount of NeuAc in the muscle tissue was measured in the placebo group and the ManNAc, NeuAc, and sialyllactose-treated groups. As shown in FIG. 6, the amount of NeuAc was significantly higher in the ManNAc, NeuAc, and sialyllactose-treated groups than in the DMRV model mice of the placebo group.

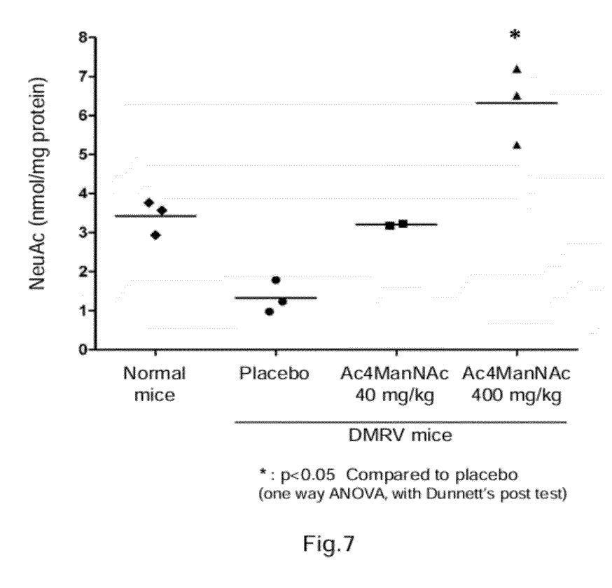

The amount of NeuAc in the muscle tissue was also measured in the 40 or 400 mg/kg Ac4ManNAc-treated group. As shown in FIG. 7, the amount of NeuAc in the muscle tissue of the DMRV model mice was significantly higher in the 400 mg/kg Ac4ManNAc-treated group than in the DMRV model mice of the placebo group.

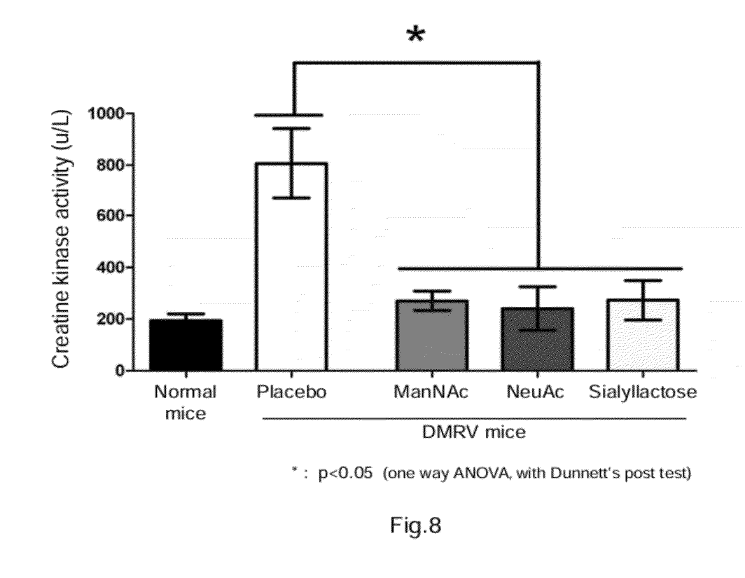

FIG. 8 shows the blood creatine kinase activity of the mice. The activity was significantly lower in the ManNAc, NeuAc, and sialyllactose-treated groups than in the placebo group.

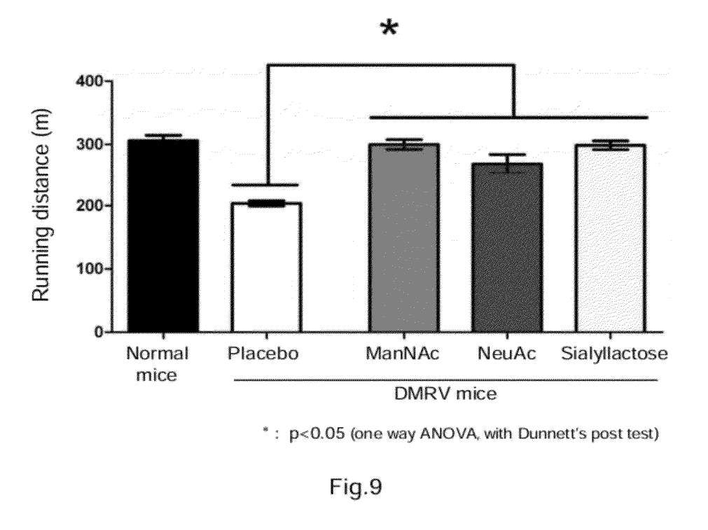

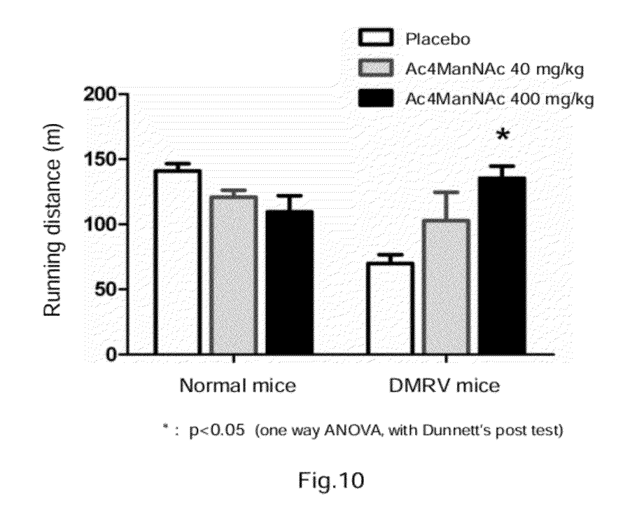

FIGS. 9 and 10 show the accumulated running distances of the mice groups in the motor ability test. The accumulated running distance was significantly higher in the ManNAc, NeuAc, and sialyllactose-treated groups than in the placebo group, indicating improved motor ability of the ManNAc, NeuAc, and sialyllactose-treated groups (FIG. 9). The accumulated running distance was significantly higher in the 400 mg/kg Ac4ManNAc-treated DMRV model mice than in the placebo group, indicating improved motor ability of the 400 mg/kg Ac4ManNAc-treated DMRV model mice (FIG. 10).

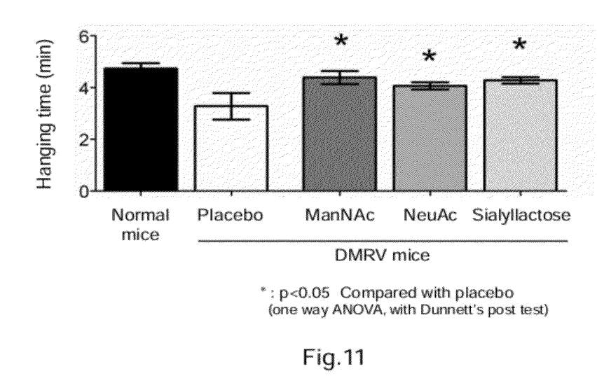

As shown in FIG. 11, the hanging time of the mice was longer in the ManNAc, NeuAc, and sialyllactose-treated groups than in the placebo group.

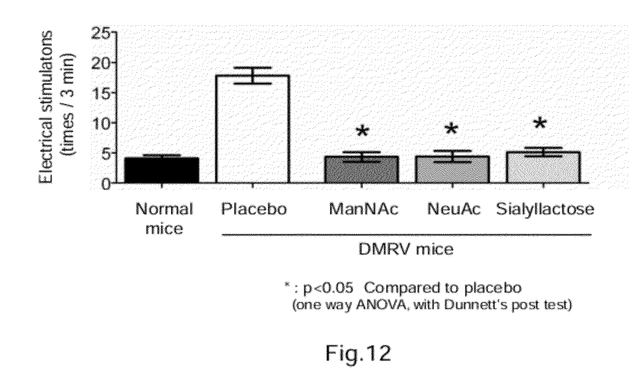

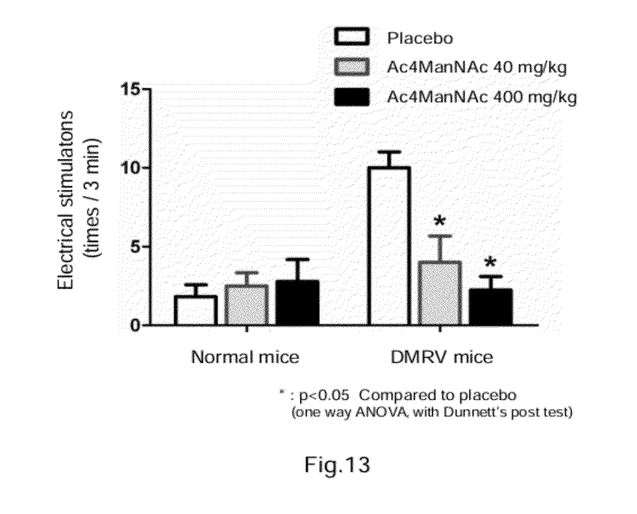

FIGS. 12 and 13 show the times of the electrical stimulation for 3 minutes in the endurance test. The times of electrical stimulation was significantly lower in the DMRV model mice of the ManNAc, NeuAc, and sialyllactose-treated groups than in the placebo group, indicating improved endurance of the ManNAc, NeuAc, and sialyllactose-treated groups (FIG. 12). The times of electrical stimulation was significantly lower in the 40 or 400 mg/kg Ac4ManNAc-treated DMRV model mice than in the placebo group, indicating improved endurance of the Ac4ManNAc-treated DMRV model mice (FIG. 13).

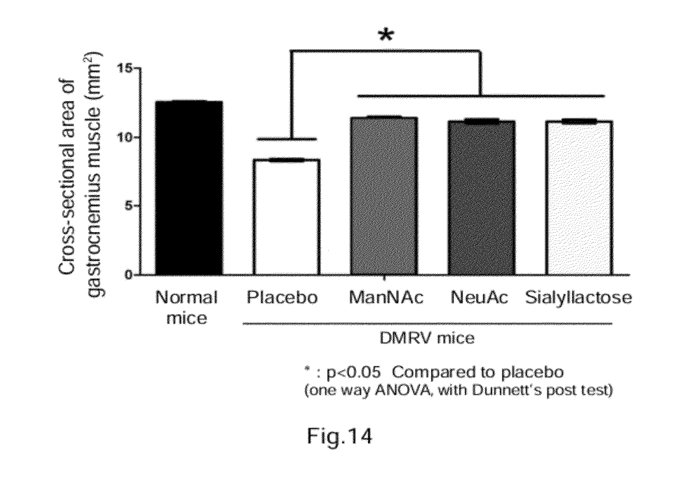

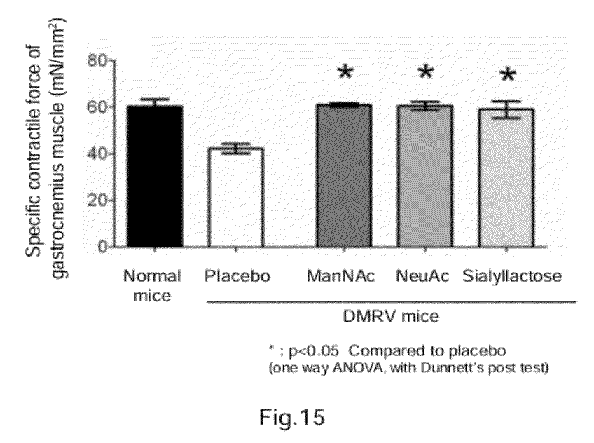

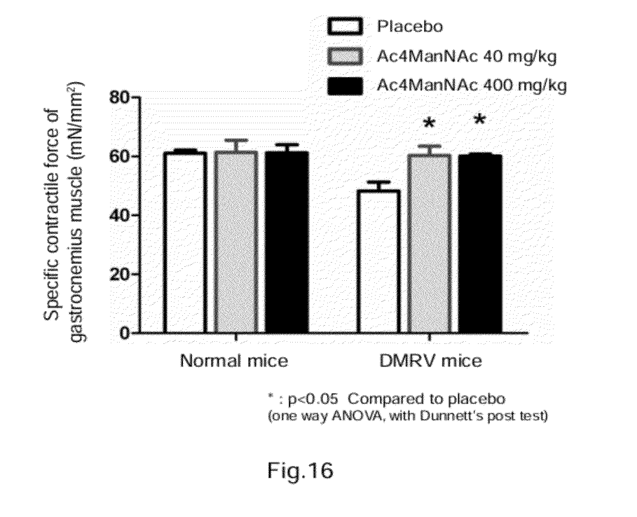

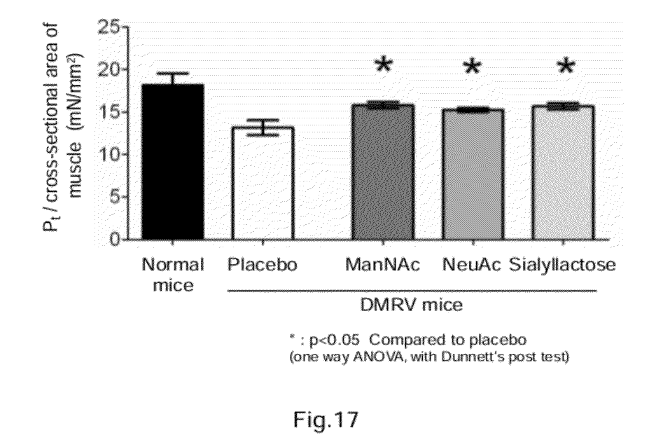

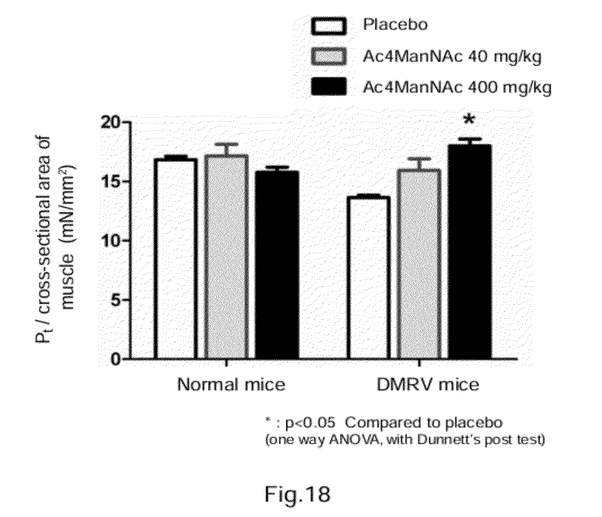

As shown in FIGS. 14 and 15, the cross-sectional area of the gastrocnemius muscle and the specific contractile force of the gastrocnemius muscle (P₀ per cross-sectional area of the muscle) were significantly higher in the DMRV model mice of the ManNAc, NeuAc, and sialyllactose-treated groups than in the placebo group. As shown in FIG. 16, the specific contractile force of the gastrocnemius muscle was significantly higher in the DMRV model mice of the 40 and 400 mg/kg Ac4ManNAc-treated groups than in the placebo group. As shown in FIGS. 17 and 18, the Pₜ per cross-sectional area of the muscle was significantly higher in the DMVR model mice of the ManNAc, NeuAc, and sialyllactose-treated groups (FIG. 17) and the 400 mg/kg Ac4ManNAc-treated group (FIG. 18) than in the placebo group.

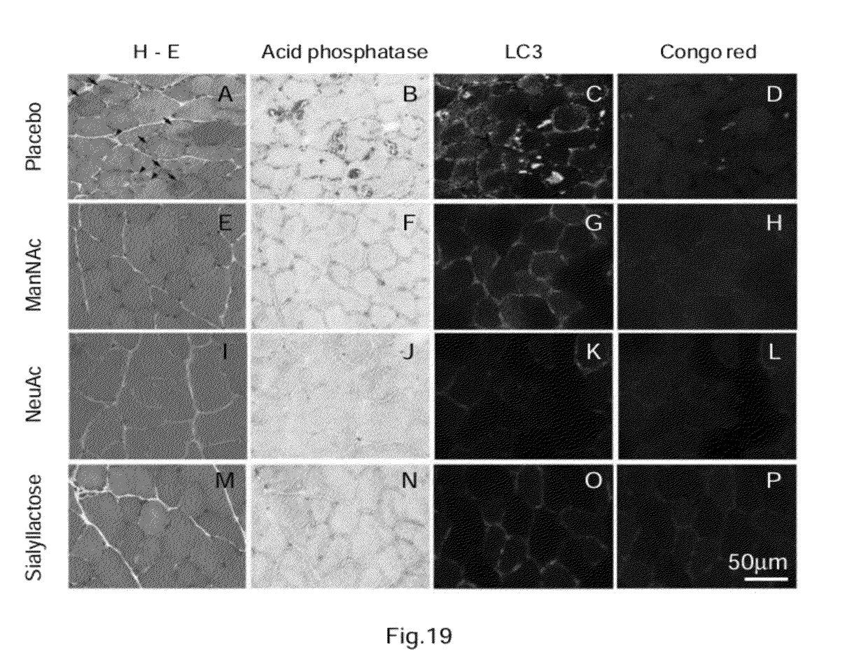

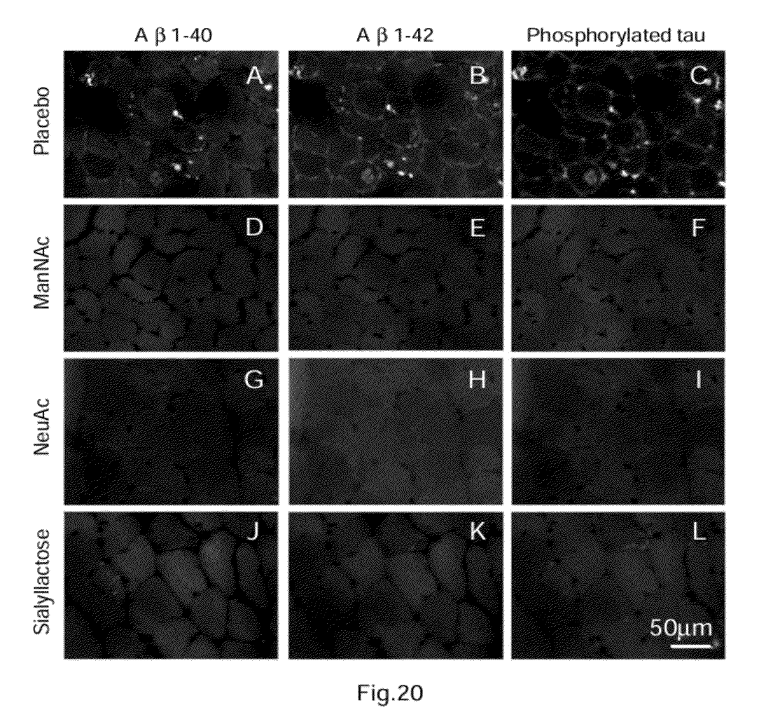

FIGS. 19 and 20 show the pathological observations of the muscle tissues of the DMRV model mice of the ManNAc, NeuAc, and sialyllactose-treated groups. As shown in FIG. 19A, the DMRV model mice of the placebo group had rimmed vacuoles (arrows) and muscle cell atrophy (arrowheads) and had many sites that were positive for the acid phosphatase activity staining in the tissue. In contrast, the ManNAc, NeuAc, and sialyllactose-treated groups had no rimmed vacuole or muscle cell atrophy (FIGS. 19E, I, and M) and were negative for the acid phosphatase activity staining (FIGS. 19F, J, and N). DMRV skeletal muscle fibers are known to have accumulated amyloid protein. Although the placebo group was labeled with the anti-amyloid antibodies (LC3, Aβ1-40, and Aβ1-42) (FIG. 19C and FIGS. 20A and B), the labeling of the ManNAc, NeuAc, and sialyllactose-treated groups was markedly reduced (FIGS. 19G, K, and O, and FIGS. 20D, E, G, H, J, and K). Likewise, only the placebo group was fluorescently labeled with congo red staining, which recognizes amyloid (FIGS. 19D, H, L, and P). Only the placebo group was also labeled with the anti-phosphorylated tau antibody (FIGS. 20C, F, I, and L). These pathological observations show that symptoms observed in the pathological tissues of the DMRV model mice, such as the formation of rimmed vacuoles, muscle cell atrophy, and the accumulation of amyloid, were alleviated in the ManNAc, NeuAc, and sialyllactose-treated groups.

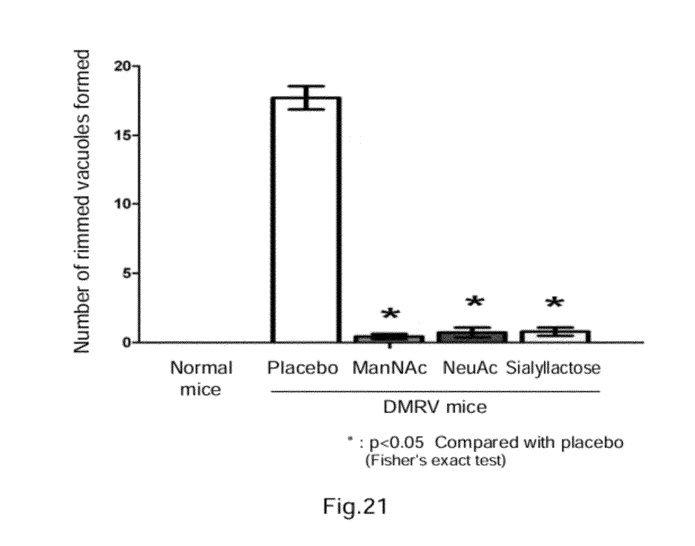

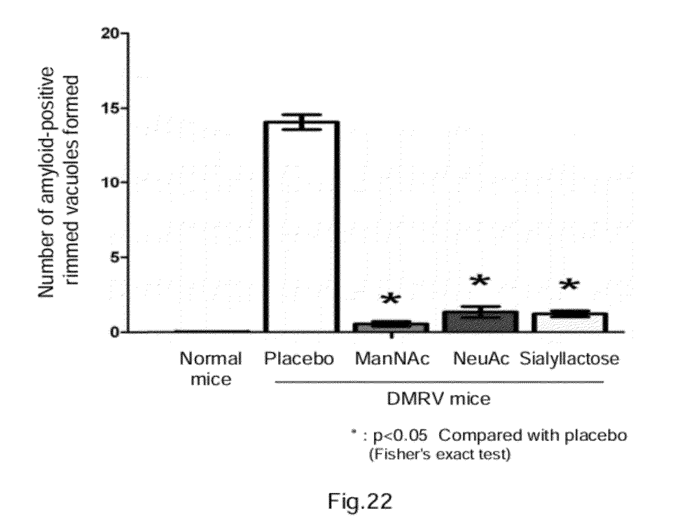

FIG. 21 shows that the number of rimmed vacuoles was significantly lower in the ManNAc, NeuAc, and sialyllactose-treated groups than in the placebo group. FIG. 22 shows that the number of amyloid positive cells was significantly lower in the ManNAc, NeuAc, and sialyllactose-treated groups than in the placebo group.

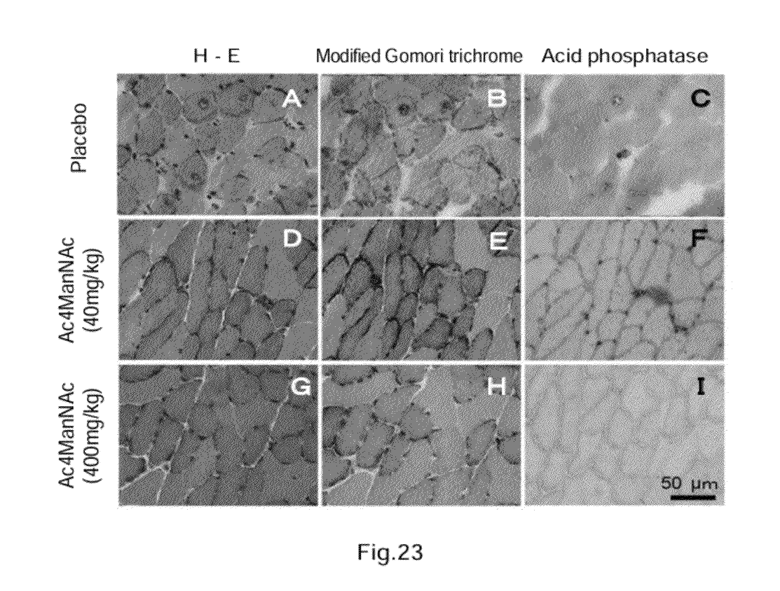

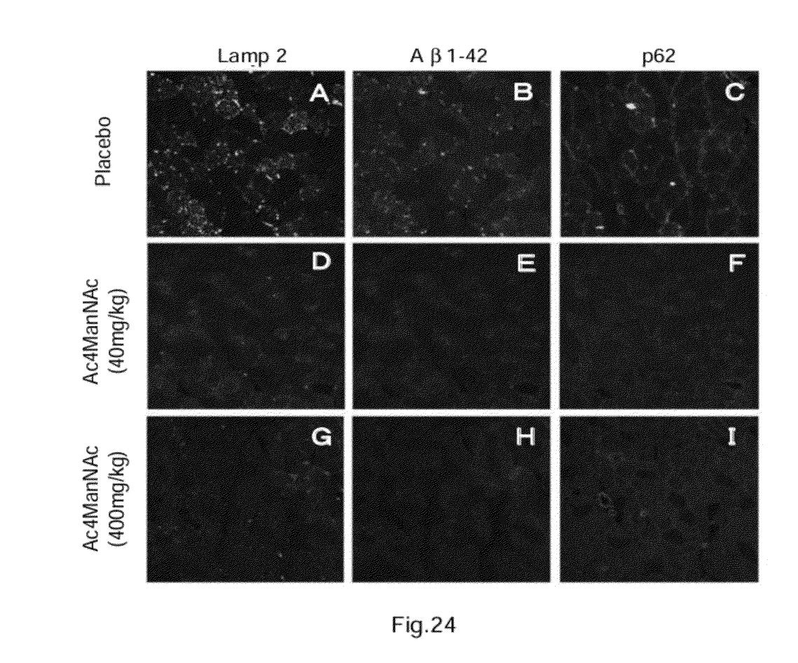

FIGS. 23 and 24 show the pathological observations of the muscle tissues of the DMRV model mice of the 40 or 400 mg/kg Ac4ManNAc-treated group. As in the observation shown in FIG. 19A, the DMRV model mice of the placebo group had rimmed vacuoles and muscle cell atrophy in the tissue stained with H-E or Gomori trichrome (FIGS. 23A and B). The DMRV model mice of the placebo group had many sites that were positive for the acid phosphatase activity staining in the tissue (FIG. 23C). The frequencies of rimmed vacuoles and muscular atrophy were much lower in the 40 mg/kg Ac4ManNAc-treated group than in the placebo group (FIGS. 23D and F). The 40 mg/kg Ac4ManNAc-treated group had dispersed sites that were positive for the acid phosphatase activity staining (FIG. 23E). In contrast, the 400 mg/kg Ac4ManNAc-treated group had no rimmed vacuole or muscle cell atrophy (FIGS. 23G and H) and was negative for the acid phosphatase activity staining (FIG. 231). The skeletal muscles of the DMRV model mice of the placebo group were positive for Lamp 2, β-amyloid (Aβ1-42), and p62 protein (FIGS. 24A, B, and C). With Lamp 2, the DMRV model mice of the 40 mg/kg Ac4ManNAc-treated group were very slightly stained, but the 400 mg/kg Ac4ManNAc-treated group was negative. The DMRV model mice of the 40 and 400 mg/kg Ac4ManNAc-treated groups were negative for Aβ1-42 and p62 protein. Thus, symptoms observed in the pathological tissues of the DMRV model mice, such as the formation of rimmed vacuoles, muscle cell atrophy, the accumulation of amyloid, and a fibrous structure, were alleviated in the Ac4ManNAc-treated groups.

These results show that the administration of ManNAc, NeuAc, an intermediate of the NeuAc biosynthetic pathway, a NeuAc derivative, a ManNAc derivative, or a NeuAc-containing compound to an individual suffering from DMRV can alleviate the symptoms of affected muscle tissues, improve motor ability of the individual, and decrease the case fatality rate.

### INDUSTRIAL APPLICABILITY

The present invention can provide a therapeutic pharmaceutical agent, a food composition, and a food additive for use in diseases caused by decrease of a GNE protein function.

What is claimed is:

1. A therapeutic pharmaceutical agent for a disease caused by decrease of a GNE protein function, comprising
one or more compounds selected from the group consisting of N-acetylneuraminic acid, an intermediate produced downstream of N-acetylmannosamine in an N-acetylneuraminic acid biosynthetic pathway, an N-acetylneuraminic acid derivative, an N-acetylmannosamine derivative, a compound containing N-acetylneuraminic acid, an N-acetylneuraminic acid degrading enzyme inhibitor, an N-acetylmannosamine degrading enzyme inhibitor, and an inhibitor of a degrading enzyme of the intermediate,
wherein the N-acetylneuraminic acid derivative has the following formula 1: 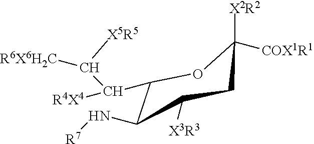
wherein X^{P} (P is an integer from 1 to 6) denotes O or S,
R^{P} (P is an integer from 2 to 6) denotes hydrogen, a lower alkanoyl, or a lower alkyl in the case that X^{P} adjacent to the R^{P} is O or denotes a lower alkanoyl or a lower alkyl in the case that X^{P} adjacent to the RP is S,
R¹ denotes hydrogen, a lower alkyl, or a lower alkanoylalkyl in the case that X¹ is O or denotes a lower alkyl or a lower alkanoylalkyl in the case that X¹ is S, and
R⁷ denotes hydrogen, a lower alkanoyl, or a lower hydroxyalkanoyl, and
the N-acetylmannosamine derivative has the following formula 2: 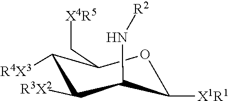
wherein X^{P} (P is an integer from 1 to 4) denotes O or S,
R^{P} (P is an integer selected from 1, 3, 4, and 5) denotes hydrogen, a lower alkyl, a lower alkanoylalkyl, or a lower alkanoyl in the case that X^{P} adjacent to the R^{P} is O or denotes a lower alkyl, a lower alkanoylalkyl, or a lower alkanoyl in the case that X^{P} adjacent to the R^{P} is S, and
R² denotes hydrogen or a lower alkanoyl.

2. The pharmaceutical agent according to claim 1, wherein the decrease of a GNE protein function is caused by a mutation of the GNE gene.

3. The pharmaceutical agent according to claim 1, wherein the disease is renal dysfunction or myopathy.

4. The pharmaceutical agent according to claim 1, wherein the intermediate is N-acetylmannosamine-6-phosphate or N-acetylneuraminic acid-9-phosphate.

5. The pharmaceutical agent according to claim 1, wherein the N-acetylneuraminic acid derivative is Ac5NeuAc or Ac5NeuAc-Me.

6. The pharmaceutical agent according to claim 1, wherein the N-acetylmannosamine derivative is Ac4ManNAc.

7. The pharmaceutical agent according to claim 1, wherein the compound containing N-acetylneuraminic acid is sialyllactose.

8. The pharmaceutical agent according to claim 1, wherein the inhibitor of the degrading enzyme of the intermediate is GlcNAcol or a GlcNAcol derivative.

9. The pharmaceutical agent according to claim 8, wherein the GlcNAcol derivative is Ac5GlcNAcol.

10. A food composition, comprising
one or more compounds selected from the group consisting of an N-acetylneuraminic acid derivative, an N-acetylmannosamine derivative, an N-acetylneuraminic acid degrading enzyme inhibitor, an N-acetylmannosamine degrading enzyme inhibitor, and an inhibitor of the degrading enzyme of an intermediate produced downstream of N-acetylmannosamine in an N-acetylneuraminic acid biosynthetic pathway,
wherein the N-acetylneuraminic acid derivative has the following formula 1: 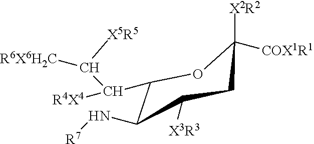
wherein X^{P} (P is an integer from 1 to 6) denotes O or S,
R^{P} (P is an integer from 2 to 6) denotes hydrogen, a lower alkanoyl, or a lower alkyl in the case that X^{P} adjacent to the R^{P} is O or denotes a lower alkanoyl or a lower alkyl in the case that X^{P} adjacent to the R^{P} is S,
R¹ denotes hydrogen, a lower alkyl, or a lower alkanoylalkyl in the case that X¹ is O or denotes a lower alkyl or a lower alkanoylalkyl in the case that X¹ is S, and
R⁷ denotes hydrogen, a lower alkanoyl, or a lower hydroxyalkanoyl, and
the N-acetylmannosamine derivative has the following formula 2: 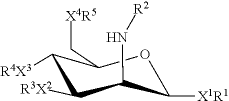
wherein X^{P} (P is an integer from 1 to 4) denotes O or S,
R^{P} (P is an integer selected from 1, 3, 4, and 5) denotes hydrogen, a lower alkyl, a lower alkanoylalkyl, or a lower alkanoyl in the case that X^{P} adjacent to the R^{P} is O,
R^{P} (P is an integer selected from 1, 3, 4, and 5) denotes a lower alkyl, a lower alkanoylalkyl, or a lower alkanoyl in the case that X^{P} adjacent to the R^{P} is S, and
R² denotes hydrogen or a lower alkanoyl.

11. The food composition according to claim 10, wherein the N-acetylneuraminic acid derivative is Ac5NeuAc or Ac5NeuAc-Me.

12. The food composition according to claim 10, wherein the N-acetylmannosamine derivative is Ac4ManNAc.

13. The food composition according to claim 10, wherein the inhibitor of the degrading enzyme of the intermediate is GlcNAcol or a GlcNAcol derivative.

14. The food composition according to claim 13, wherein the GlcNAcol derivative is Ac5GlcNAcol.

15. A food, comprising a food composition according to claim 10.

16. A food additive, comprising
one or more selected from N-acetylneuraminic acid, an intermediate produced downstream of N-acetylmannosamine in an N-acetylneuraminic acid biosynthetic pathway, and a compound containing N-acetylneuraminic acid,
wherein the N-acetylneuraminic acid, the intermediate produced downstream of N-acetylmannosamine in the N-acetylneuraminic acid biosynthetic pathway, and the compound containing N-acetylneuraminic acid are compounds purified from natural products or chemically synthesized compounds.

17. The food additive according to claim 16, wherein the total amount of the N-acetylneuraminic acid, the intermediate produced downstream of N-acetylmannosamine in the N-acetylneuraminic acid biosynthetic pathway, and the compound containing N-acetylneuraminic acid is 50% or more of the food additive.

18. A method for producing a food containing a food additive, comprising the addition of a food additive according to claim 16.

19. A food containing a food additive, produced by the method according to claim 18.
